(19) United States
(12) Patent Application Publication
(10) Pub. No.: US 2003/0190378 A1
(43) Pub. Date: Oct. 9, 2003

(54) Extract of processed Panax genus plant, the preparation method thereof, and compositions containing the same

(75) Inventors: Dong-Hyun Kim, Seoul (KR); Eun-Ah Bae, Seoul (KR); Myung-Joo Han, Seoul (KR); Min-Kyung Choo, Seoul (KR); Eun-Kyung Park, Seoul (KR); Jeong-Hill Park, Seoul (KR)

Correspondence Address: FOLEY AND LARDNER, SUITE 500, 3000 K STREET NW, WASHINGTON, DC, 20007, US

(73) Assignee: Ginseng Science Inc.

(21) Appl. No.: 10/345,209

(22) Filed: Jan. 16, 2003

(30) Foreign Application Priority Data
Apr. 8, 2002 (KR) 10-2002-0018856
Dec. 21, 2002 (KR) 10-2002-0082055

(51) Int. Cl. A61K 35/78; A61K 31/366

(52) U.S. Cl. 424/728; 514/460

(57) ABSTRACT

The present invention relates to an extract of processed Panax genus plant, the preparation thereof and compositions containing the same having anticancer or anti-allergic activity. More particularly, the present invention relates to a processed ginseng product with enhanced pharmacological effects due to serial treatment i.e., acid-treatment or heat-treatment of a Panax genus plants and subsequent bio-converting treatment such as lactic fermenting and intestinal-bacterial fermenting process so as to make a ratio of ginsenoside (Rk₂+Rh₃+protopanaxadiol+20-dehydroprotopanaxadiol) to (Rg₃+Rg₅+Rk₁) of above 0.1. The extract of processed Panax genus plant in the present invention has inhibitory effect for cancer or allergic diseases and it is useful in the prevention or treatment of cancer or allergic diseases.

The present invention relates to an extract of processed Panax genus plant, the preparation thereof and compositions containing the same having anticancer or anti-allergic activity. More particularly, the present invention relates to a processed ginseng product with enhanced pharmacological effects due to serial treatment i.e., acid-treatment or heat-treatment of a Panax genus plants and subsequent bio-converting treatment such as lactic fermenting and intestinal-bacterial fermenting process so as to make a ratio of ginsenoside (Rk₂+Rh₃+protopanaxadiol+20-dehydroprotopanaxadiol) to (Rg₃+Rg₅+Rk₁) of above 0.1. The extract of processed Panax genus plant in the present invention has inhibitory effect for cancer or allergic diseases and it is useful in the prevention or treatment of cancer or allergic diseases.

### DESCRIPTION

1. Field of the Invention

The present invention relates to an extract of processed Panax genus plant, the preparation method thereof and compositions containing the same having anti-cancer and anti-allergic activity. More particularly, the present invention relates to a processed ginseng product and the extract thereof with enhanced pharmacological effects due to serial treatment i.e., acid-treatment or heat-treatment of a Panax genus plants and subsequent bio-converting treatment such as lactic fermenting and intestinal-bacterial fermenting process so as to make a ratio of ginsenoside (Rk₂+Rh₃+protopanaxadiol+20-dehydroprotopanaxadiol) to (Rg₃+Rg₅+Rk₁) of above 0.1.

2. Background of the Invention

It is known that there are many genus of Panax genus plants belonged to Araliaceae, for example, Panax ginseng distributed or cultivated in far-eastern Asia region, Panax quinquefolia in America and Canada, Panax notoginseng in China, Panax trifolia in eastern region of north America, Panax japonica in Japan, China and Nepal, Panax pseudoginseng in Nepal, Panax vietnamensis in Vietnam, Panax elegatior, Panax wangianus and Panax bipinratifidus etc.

Hitherto, a ginseng has been widely known as a representative nutritive tonic agent. Recently, various scientific studies on the chemical constituents and pharmacological effects of the ginseng have been reported so that the secret pharmacological effects are paid attention with modern scientific approaches. Until now, it has been known that the ginseng has various pharmacological effects such as prevention of aging, anti-arteriosclerosis, treatment of hyperlipidemia, treatment of hepatic insufficiency, improvement of liver function, protection of radiation injury, immune enhancement, improvement of cerebral function, anti-thrombotic, anti-stress, anti-diabetic, anti-hypertensive, anti-tumor effects, etc.

It has been known that the main constituents of Panax genus plant are dammarane saponins. Ginsenosides Rb₁, Rb₂, Rc, Rd, Rg₁, and Re are main saponins of Panax ginseng. Their activities are different from each other in accordance with their chemical structures.

There have been many attempts to process or modify Panax genus plants so as to increase their pharmacological potency, in particular, to modify the structure of ginsenoisides therein.

Korean Patent Publication No. 10-1996-017670 issued on May. 23, 1996, discloses a process for preparing a processed ginseng prepared by subjecting hot temperature treatment containing high contents of ginsenoside Rg₃ and Rg₅ so as to obtaining processed ginseng having improved potency differing from original form of ginseng.

Korean Patent Publication No. 10-1996-004217 issued on Feb. 22, 1996, discloses a process for mass production of saponin metabolites such as compound K from ginseng saponins using intestinal-bacteria.

However, there have been no disclosure or suggestion about a process for preparing processed Panax genus plant prepared by serial treatment comprising acid or heat treatment or their combination thereof and subsequent fermentation with lactic-acid bacteria or intestinal-bacteria so as to chemically change their saponin components resulting in a ratio of ginsenoside (Rk₂+Rh₃+protopanaxadiol+20-dehydroprotopanaxadiol) to (Rg₃+Rg₅+Rk₁) of above 0.1.

Recently, however, the process wherein a ginseng is heat-treated at a high temperature has been partially tried out. For example, Japanese Patent Application Laid-open No. (Sho) 62-158490 discloses a process for preparing a ginseng tissue-cultivate having a high ginsenoside Rh content which comprises heat-treating the ginseng tissue-cultivate at a temperature of 110 to 160° C. However, it has the drawback that since the ginseng tissue-cultivate was used instead of ginseng as it is, the processed ginseng product has not a shape of an original ginseng and that tissue cultivate of the ginseng is different from the naturally cultivated original ginseng with respect to the various components and pharmacological effects Korean Journal of Pharmacognosy 16, page 171 (1985) and that the process is also complex and noneconomic due to the tissue-cultivating procedure. In addition, although a process wherein a ginseng was heat-treated at a high temperature was partially tried out, the process was merely employed in a process for manufacturing cosmetics or tea, and the study relating to the pharmacological effects of heat-treated ginseng was not carried out.

The inventors of the present invention have intensively carried out the scientific investigation concerning components and pharmacological effects of a ginseng, in particular a processing method of a ginseng and physiological activity of the processed ginseng. As a result of the investigation, the inventors have discovered that because the contents of the trace components present in Panax genus plant are significantly increased as well as novel components are produced by serial treatment comprising acid or heat treatment or their combination thereof, followed by fermentation with lactic-acid bacteria or intestinal-bacteria, the extract of plant has novel components which ever been found yet till now and shows substantially enhanced pharmacological effects, and they have finally completed the present invention.

### SUMMARY OF THE INVENTION

Accordingly, it is an object of the present invention to provide a Panax genus plant or the extract thereof comprising a ratio of ginsenoside (Rk₂+Rh₃+protopanaxadiol+20-dehydroprotopanaxadiol) to (Rg₃+Rg₅+Rk₁) of above 0.1.

And, another object of the present invention is to provide a Panax genus plant or the extract thereof obtained by the steps essentially comprising acid or heat treating or their combination thereof the plant material belonged to Panax genus and subsequent fermentation treating with lactic-acid bacteria or intestinal-bacteria.

An additional object of the present invention is to provide a process for preparing above described plant and the extract thereof.

An additional object of the present invention is to provide pharmaceutical compositions comprising above extract or the saponin compounds isolated therefrom as an active ingredient in an amount effective to treat or prevent cancer or allergic disease of human or mammal, together with a pharmaceutically acceptable carrier.

An additional object of the present invention is to provide a method for treating or preventing cancer or allergic disease in a mammal comprising administrating to said mammal an effective amount of above extract or the saponin compounds isolated therefrom, together with a pharmaceutically acceptable carrier thereof.

An additional object of the present invention is to provide a use of above extract or the saponin compounds isolated therefrom, in the manufacture of a medicament for the treatment of cancer or allergic disease.

### DETAILED DESCRIPTION

In accordance with the present invention, the present invention provides a Panax genus plant or the extract thereof comprising a ratio of (Rk₂+Rh₃+protopanaxadiol+20-dehydroprotopanaxadiol) to (Rg₃+Rg₅+Rk₁) of above 0.1, preferably, above 0.2, more preferably above 0.5.

Above described protopanaxadiol (PPD) and dehydroprotopanaxadiol (DHPPD) comprise their isomers, enantiomers and diasteromers, i.e., (20S) PPD, (20R) PPD and 20(21)-DHPPD, 20(22)-DHPPD.

The present invention also provides a processed Panax genus plant or the extract thereof obtained by the steps essentially comprising acid or heat treating or their combination thereof the plant material belonged to Panax genus, and subsequent fermentation treating with lactic-acid bacteria or intestinal-bacteria.

Above described plant or extract can be prepared by following steps:

1. 1^{st} Step:

1^{st} step is to subject following acid or heat treatment step or the combinations thereof to plant material as follows;

(1) Acid Treatment Step

Specifically, at the 1₁st step, dried plant material belonged to Panax genus for examples, the root of Panax ginseng, is subjected to following acid treatment; for example, about 1 to 50 times, preferably 20 to 20 times w/v % of 0.01 to 50%, preferably, 0.1 to 10% acidic component, preferably, acetic acid, citric acid, lactic acid or acidic tasting food such as Schisandra chinensis, is added to the plant material and then is subject to incubation at the temperature ranging from 20 to 80° C., preferably 40 to 70° C. for a period ranging from 1 to 48 hrs, preferably, 3 to 12 hrs; to organic solvent such as butanol, methanol, ether, ethyl acetate is added thereto and then subject to extraction to obtain organic solvent soluble extract; the extract is neutralized with base finally to obtain chemically acidified Panax genus extract.

(2) Heat Treatment Step

As another initial step to obtain the present invention, heat treatment process can be employed, i.e., dried plant material belonged to Panax genus is subjected to following heat treatment; for example, the plant material is treated at a temperature ranging from 110 to 180° C., preferably, 120 to 140° C. for a period ranging from 0.5 to 20 hours, preferably 2 to 5 hours. The heating time varies depending on the heating temperature. The lower heating temperature requires the longer heating time. The heating procedure may be carried out by using a hot air, water vapor, nitrogen, helium, carbon dioxide or mixed gas thereof. In order to increase the efficiency, the heating process may be preferably performed in an airtight container such as autoclave. Alternatively, a small amount of water may be added to the container; otherwise, the ginseng may be preferably soaked in water and then heated in a closed container.

The ginseng thus processed may be dried at a lower temperature than the heating temperature of the preceding procedure, i.e., a normal temperature to 80° C. by a known manner to obtain a dried processed ginseng, or it may be further processed to obtain a powdered ginseng, if necessary.

Alternatively, the processed ginseng may be extracted using a known manner to obtain a processed ginseng extract. Specifically, the processed ginseng is extracted by using a solvent, and then the solvent is removed by subjecting to concentration or freeze-drying treatment to obtain a processed ginseng extract as dried powders.

The solvent which may be employed herein includes a water, lower alcohol such a methanol, ethanol, etc., lower ketone such as acetone, methylethylketone, etc., supercritical fluid or mixed solvent thereof.

The plant material which may be employed includes, but are limited to, Panax genus plant itself such as a fresh ginseng, a white ginseng and red ginseng, a fine root of ginseng or ginseng leaves or extracts thereof, which can be used as it is, finely divided or powdered, processed product thereof and their by-product which comprise dammarane type saponin, preferably, the root, stem, petal, leaf, fruit of Panax ginseng, Panax quinquefolia, Panax notoginseng, Panax trifolia, Panax japonica, Panax pseudoginseng, Panax vietnamensis, Panax elegatior, Panax wangianus, Panax bipinratifidus and Panax angustifolium and their tissue cultivates and the extract thereof.

Above (1) and (2) processes can be subjected to plant material, independently or in a combination manner prior to following 2^{nd} step.

2. 2^{nd} Step: Fermentation Step

The extract obtained from 1^{st} step is subsequently subject to following bioconversion process such as fermentation with lactic acid or intestinal-bacteria as follows:

For example, lactic acid bacteria or intestinal-bacteria is added to the extract obtained from 1^{st} step and incubated at a temperature ranging from 20 to 50° C., preferably, 25 to 40° C. for a period ranging from 8 hours to 8 days, preferably 24 hours to 3 days to obtain treated extract with bacteria.

The incubation time varies depending on the genus of used bacteria.

The lactic acid bacteria which may be employed includes any one which can metabolize ginsenoside Rg₃, Rg₅ and Rk₁, to ginsenoside Rh₂, Rh₃, Rk₂, protopanaxadiol (PPD) and 20-dehydroprotopanaxadiol (DHPPD), preferably, lactic acid bacteria belonged to Bifidobacterium genus, more preferably, at least one or the mixture thereof selected from the group consisting of Bifidobacterium infantis, Bifidobacterium bifidum, Lactobacillus lactis, Clostridium butyricum, Bifidobacterium K-103, Bifidobacterium K-506, Bifidobacterium K-513, Bifidobacterium K-525, Bifidobacterium KK-1 and Bifidobacterium KK-2 (disclosed in Arch. Pharm. Res., 21, p54-61, 1988).

The intestinal-bacteria which may be employed includes any one which can metabolize ginsenoside Rg₃, Rg₅ and Rk₁, to ginsenoside Rh₂, Rh₃, Rk₂, protopanaxadiol (PPD) and dehydroprotopanaxadiol (DHPPD), preferably, intestinal-bacteria belonged to Bacterioides, Fusobacterium and Eubacterium genus, more preferably, at least one or the mixture thereof selected from the group consisting of Bacteriodes JY-6 (disclosed in Biol. Pharm. Bull., 23, pp1481-1485, 2000), Bacteriodes stercoris, Fusobacterium K-60 (disclosed in Biol. Pharm. Bull., bid.) and Eubacterium L-8, (disclosed in Biol. Pharm. Bull., bid.).

Further to above described steps, to isolate the saponin fractions or the saponin compounds from the extract obtained from above 2^{nd} step, following processes can be adopted.

3. 3^{rd} Step: Isolation Process

To isolate pharmacologically active fractions or saponin compounds from the extract prepared by 2^{nd} step, lower alcohols such as alcohol water, methanol and ethanol can be used to extract or isolate the fractions or compounds from the extract obtained from 2^{nd} step as an appropriate solvent.

Additionally, the active ingredient can be extracted or isolated by subjecting special extraction method such as supercritical fluid extraction (SFE) to obtain partially purified saponin fractions and further, Silica gel column chromatographic method to isolate various saponins thereby.

Subsequent to above step, following processes such as drying process by lyophilization, agitation or dilution process for drink or food, can be adopted in addition to above steps, if necessary.

Following processes can be selected either or both according to the final product forms of the present invention.

4. 4^{th} Step: Drying process

(1) Above extract of Panax genus plant obtained in Step 2 or 3, is concentrated in vacuo and then dried such as lyophilization or spray drying.

(2) Above extract of Panax genus plant obtained in Step 2 or 3, is centrifuged to remove its impurities and precipitate and the supernatant is concentrated in vacuo and then dried such as lyophilization or spray drying.

Through above 1^{st} step to 2^{nd} step processes, saponins such as ginsenoside Rb₁, Rb₂, Rc, Rd etc contained in plant material, is transformed into chemically modified ginsenosides such as ginsenoside Rg₃, Rg₅, Rk₁ etc through the acid treatment or heat treatment in step 1 and then the sugar moiety at the position 3 in modified saponins is further degraded to form further modified saponins comprising ginsenoside Rk₂, Rh₂, Rh₃, PPD, DHPPD.

In particular, the processed ginseng product according to the present invention wherein a ratio of ginsenoside (Rk₂+Rh₃+protopanaxadiol+20-dehydroprotopanaxadiol) to (Rg₃+Rg₅+Rk₁) of above 0.1, shows superior physiological activities to the prior processed ginseng product in which the contents of ginsenosides Rk₂, Rh₂, Rh₃, PPD and DHPPD are near 0.

Therefore, the present invention also provides the process for preparing above described plant or the extract thereof essentially comprising the steps of acid or heat treating or their combination thereof the plant material belonged to Panax genus and subsequent fermentation step treated with lactic-acid bacteria or intestinal-bacteria.

The present invention additionally provides a process for preparing of pharmacologically active fractions or saponin compounds, comprising the steps of; subjecting the extract prepared by above step 2 to extraction or isolation by lower alcohols to obtain purified fraction; and additionally, subjecting the fraction to Silica gel column chromatography to isolate saponin compounds.

Above described lower solvent includes methanol, ethanol and butanol.

The present invention also provides pharmaceutical compositions comprising above described extract or saponin compounds prepared by above processes as an active ingredient in an amount effective to treat or prevent human or mammal diseases in particular, cancer or allergic disease, together with a pharmaceutically acceptable carrier.

Specifically, the present invention provides pharmaceutical compositions comprising above described extract or saponin compounds selected from the group consisting of ginsenoside Rb₁, Rb₂, Re, Rg₁, Rf, F1, F4, Rh₁, Rg₅, 20(R)-ginsenoside Rg₃, 20(S)-ginsenoside Rg₃, 20(S)-ginsenoside Rh₂, 20(R)-ginsenoside Rh₂, protopanaxadiol and the mixture thereof as an active ingredient in an amount effective to treat or prevent human or mammal cancer or allergic diseases, together with a pharmaceutically acceptable carrier.

The above-described cancer comprises liver cancer, lung cancer, skin cancer, ovarian cancer, blood cancer, uterine cancer and the like and the above-described allergic diseases comprise rhinitis, dermatitis, asthma, autoimmune deficiency disease and the like.

The present invention also provides a method for treating or preventing cancer or allergic disease in a mammal comprising administrating to said mammal an effective amount of above described extract and the saponin compounds isolated therefrom and pharmaceutically acceptable carrier thereof.

The present invention also provides a use of above described extract and the saponin compounds isolated therefrom in the manufacture of a medicament for the treatment of cancer or allergic disease.

Specifically, above described saponin compounds comprise ginsenoside Rb₁, Rb₂, Re, Rg₁, Rf, F1, F4, Rh₁, Rg₅, 20(R)-ginsenoside Rg₃, 20(S)-ginsenoside Rg₃, 20(S)-ginsenoside Rh₂, 20(R)-ginsenoside Rh₂, and protopanaxadiol.

The inventive composition may additionally comprise conventional carrier, adjuvants or diluents in accordance with a using method. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents which are commonly used to produce an injection. Suitable examples of the carriers include physiological saline, polyethylene glycol, ethanol, vegetable oils, isopropyl myristate, etc., but are not limited to them. For topical administration, the compounds of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or composition varies depending on the condition and the weight of the subject, severity, drug form, route and period of administration, and may be chosen by those skilled in the art. However, in order to obtain desirable effects, it is generally recommended to administer at the amount ranging 0.01-10 g/kg, preferably, 1 to 5 g/kg by weight/day of the inventive extract or compounds of the present invention. The dose may be administered in single or divided into several times per day. In terms of composition, the complex herbal composition should be present between 0.01 to 80% by weight, preferably 0.5 to 50% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

The present inventors demonstrated that anticancer and anti-allergic effect of present composition is equivalent to or more potent than that of conventional anti-allergy drug by accomplishing in vitro and in vivo experiment, e.g., cancer cell line and rat mast cell assay test using RBL-2H3 cell, passive cutaneous anaphylaxis (PCA) in mice model test, inhibition test of allergic reaction in the back of mice administrated with Dinitrophenol-human serum album (DNP-BSA), protection effect against allergic inflammation by IgE serum in mice, therefore, it is confirmed that above described composition is very useful in the prevention or treatment of cancer and allergic disease.

The composition of the present invention provides prevention for cancer and allergic disease, thus it is very useful for patients susceptible with various cancers and allergic disease.

Accordingly, it is another object of the present invention to provide a health care food comprising above described extract prepared by above processes and a sitologically acceptable additive to prevent cancer or allergic diseases.

Above described composition therein can be added to food, additive or beverage for prevention of cancer or allergic diseases. For the purpose of preventing cancer or allergic diseases, wherein, the amount of above described extract or compound in food or beverage may generally range from about 0.1 to 15 w/w %, preferably 1 to 10 w/w % of total weight of food for the health food composition and 1 to 30 g, preferably 3 to 10 g on the ratio of 100 ml of the health beverage composition.

Providing that the health beverage composition of present invention contains above described extract or compound as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 ml of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition.

Examples of addable food comprising aforementioned extract therein are various food, beverage, gum, vitamin complex, health improving food and the like.

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

### Comparative Example 1

Preparation of the Extract of Non-Processed Panax Genus Plant

60% ethanol(v/v %) was added to each air-dried and sliced 20 g of Panax ginseng root, Panax quinquefolia root and Panax notoginseng root and refluxed for three hours and concentrated in vacuo to obtain 4.5, 4.0 and 3.7 g of the extract of Panax ginseng root, Panax quinquefolia root and Panax notoginseng root respectively.

### Comparative Example 2

Preparation of the Extract of Acid Treatment Panax Genus Plant

1000 ml of water containing 0.1% lactic acid (v/v %) was added to each air-dried and sliced 20 g of Panax ginseng root, Panax quinquefolia root and Panax notoginseng root and incubated at 60° C. for 5 hours and the cultivates was subjected to the solvent extraction with butanol to obtain 2.5, 2.8 and 3.2 g of acid treated extract of Panax ginseng root, Panax quinquefolia root and Panax notoginseng root respectively.

### Comparative Example 2

Preparation of Acid-Treated Extract of Panax Genus Plant

1000 ml of water containing 0.1% lactic acid (v/v %) was added to each air-dried and sliced 20 g of Panax ginseng root, Panax quinquefolia root and Panax notoginseng root and incubated at 60° C. for 5 hours and the cultivates was subjected to the solvent extraction with butanol to obtain 2.5, 2.8 and 3.2 g of acid treated extract of Panax ginseng root, Panax quinquefolia root and Panax notoginseng root respectively.

### Comparative Example 3

Preparation of Heat-Treated Extract of Panax Genus Plant

Air-dried and sliced 100 g of Panax ginseng root, Panax quinquefolia root and Panax notoginseng root was placed into an autoclave and then was heated by steaming at 130° C. for 3 hours. 60% ethanol (v/v %) was added thereto and then refluxed for three hours to obtain 42, 35 and 37 g of heat-treated extract of Panax ginseng root, Panax quinquefolia root and Panax notoginseng root respectively.

### Example 1

Preparation of Processed Extract of Panax Genus Plant

Each heat-treated extract prepared by Comparative Example 3 in an amount equivalent to 1 g of plant material, i.e., Panax ginseng root, Panax quinquefolia root and Panax notoginseng root was dissolved in 20 ml of distilled water. 100 mg of Fusobacterium K-60 (wet weight) was added thereto and then was incubated at 37° C. for 72 hours. The incubates was centrifuged and the supernatant was concentrated and dried to obtain 550, 530 and 430 mg of processed extract of Panax ginseng root, Panax quinquefolia root and Panax notoginseng root respectively.

### Example 2

Preparation of Processed Extract of Panax Genus Plant

Each heat-treated extract prepared by Comparative Example 3 in an amount equivalent to 1 g of plant material, i.e., Panax ginseng root, Panax quinquefolia root and Panax notoginseng root was dissolved in 20 ml of distilled water. 50 mg of Bifidobacterium K-506 (Disclosed in Arch. Pharm. Res., 21, pp54-61, 1988), 50 mg of Bifidobacterium K-103 (Disclosed in Arch. Pharm. Res., 21, pp54-61, 1988) and 50 mg of Bifidobacterium KK-1 was added thereto and then was incubated at 37° C. for 72 hours. The incubates was centrifuged and the supernatant was concentrated and dried to obtain 580, 450 and 410 mg of processed extract of Panax ginseng root, Panax quinquefolia root and Panax notoginseng root respectively

### Example 3

Preparation of Processed Extract of Panax Genus Plant

Each acid-treated extract prepared by Comparative Example 2 in an amount equivalent to 1 g of plant material, i.e., Panax ginseng root, Panax quinquefolia root and Panax notoginseng root was dissolved in 20 ml of distilled water. 50 mg of Bacterioides JY-6, 50 mg of Eubacterium L-8 and 50 mg of Bacteriodes stercoris was added thereto and then was incubated at 37° C. for 48 hours. The incubates was centrifuged and the supernatant was concentrated and dried to obtain 580, 630 and 450 mg of processed extract of Panax ginseng root, Panax quinquefolia root and Panax notoginseng root respectively.

### Example 4

Preparation of Processed Extract of Panax Genus Plant

Acid-treated extract prepared by Comparative Example 2 in an amount equivalent to 1 g of Panax ginseng root was dissolved in 20 ml of distilled water. 50 mg of Bifidobacterium K-506 (Disclosed in Arch. Pharm. Res., 21, pp54-61, 1988) and 50 mg of Bifidobacterium K-103 (Disclosed in Arch. Pharm. Res., 21, pp54-61, 1988) was added thereto and then was incubated at 37° C. for 72 hours. The incubates was centrifuged and the supernatant was concentrated and dried to obtain 430 mg of processed extract of Panax ginseng root.

### Example 5

Preparation of Processed Extract of Panax Genus Plant

Non-processed extract prepared by Comparative Example 1 in an amount equivalent to 1 g of Panax ginseng root was dissolved in 20 ml of distilled water containing 1% citric acid and incubated at 60° C. for 5 hours. The pH of cultivates was adjusted with NaOH or Calcium glucuronic acid to 6.8-7.0 and centrifuged to obtain its supernatant. 50 mg of Bifidobacterium K-506 and 50 mg of Bifidobacterium KK-2 (wet weight) was added thereto and then was incubated at 37° C. for 48 hours. The incubates was centrifuged and the supernatant was concentrated and dried to obtain 350 mg of processed extract of Panax ginseng root.

### Example 6

Preparation of Processed Extract of Panax Genus Plant

1 g of sliced Panax ginseng leaves was dissolved in 200 ml of MeOH, was refluxed for 3 hours and then the solvent was removed under reduced pressure. The remaining residue was suspended in distilled water and extracted with ether to remove ether soluble compounds. Remaining water layer was extracted with butanol and concentrated to obtain butanol soluble fraction. The butanol soluble fraction was heated at 130° C. for 3 hours and then 20 ml of distilled water was added to dissolve the solution. 100 mg of fresh human intestinal-bacterial colony was added thereto, and then was incubated at 37° C. for 48 hours. The incubates was centrifuged and the supernatant was extracted with 50 ml of butanol, concentrated in vacuo and dried to obtain 100 mg of processed extract of Panax ginseng leaves.

### Example 7

Preparation of Processed Extract of Panax Genus Plant

10 l of MeOH was added to 1 kg of dried 6 year old white ginseng, and extracted five times at room temperature for 48 hours, and concentrated in vacuo to obtain 50 g of methanol soluble extract (yield: 5%). 300 ml of distilled water was added thereto and then suspended to make its suspension solution. 500 ml of butanol was added thereto and then fractioned three times to obtain 15 g of saponin fraction.

1000 ml of distilled water containing 0.1% lactic acid was added to 15 g of above saponin fraction and incubated at 60° C. for 5 hours to obtain acid treated ginseng. The incubates were neutralized with NaOH and diluted with optimum amount of water. 15 g of Bifidobacterium KK-1 (No. of Subscription: KCCM 10364) and 15 g of Bifidobacterium KK-2 (No. of Subscription: KCCM 10365) was added thereto and then was incubated at 37° C. for 72 hours. The incubates were extracted with 1000 ml of butanol twice, concentrated in vacuo and dried to obtain 8.5 g of processed saponin fraction, the fraction was dissolved in distilled water and fresh human intestinal-bacterial colony was added thereto, and then was incubated at 37° C. for 48 hours. The incubates was centrifuged and the supernatant was extracted with 50 ml of saturated butanol, concentrated in vacuo and dried to obtain 100 mg of processed extract. 8.5 g of saponin fraction was subjected to Silicagel column chromatography (3.5×60 cm, developing solvent: CHCl₃-MeOH=10:1) to give 100 mg of 20(S)-ginsenoside Rg₃, 50 mg of 20(R)-ginsenoside Rg₃, 10 mg of ginsenoside Rg₅, 10 mg of ginsenoside Rk₁, 70 mg of 20(S)-ginsenoside Rh₂, 8 mg of 20(R)-ginsenoside Rh_{2, 15} mg of ginsenoside Rh₃, 10 mg of ginsenoside Rk₂, 10 mg of 20(S)-protopanaxadiol, 2 mg of 20(R)-protopanaxadiol, 5 mg of 20-dehydroprotopanaxadiol, 15 mg of ginsenoside Rh₁, 12 mg of protopanaxatriol.

### Experimental Example 1

Content Analysis Experiment

Each extract obtained from above Comparative Example 1, 2, 3 and Example 1, 2, 3 in an amount equivalent to 500 mg of plant material was suspended with distilled water and extracted with n-BuOH. The butanol soluble layer was concentrated in vacuo and remaining residue was dissolved in 5 ml of MeOH. The solution was subjected to membrane filtration and injected to HPLC to determine the amount of saponin components therein.

The analytical method was slightly modified with the methods disclosed in the literature (Kwon et al., J. Chromatography, A 921, pp335-339, 2001) as following:

Column: LiChrosorb RP-18

Elution solvent: A═H₂0, B═CH₃CN, gradient elution

0 min (B 15%); 10 min (B 34.5%); 5 min (B 47.5%); 40 min (B 80%); 50 min (B 100%)

Flowrate: 1 ml/min.

Detector: Evaporative Light Scattering Detector (ELSD)

The results thus obtained are shown in following Table 1.

Table 1: The variation of relative amount of saponin component according to processing method

| | Rk₂ + | | | | | |
|---|---|---|---|---|---|---|
| Sample¹⁾ | Rh₃ | PPD²⁾ | DHPPD³⁾ | Rg₃ | Rg₅ + Rk₁ | Ratio⁴⁾ |
| Comparative | | | | | | |
| Example 1 | | | | | | |
| A | — | — | — | — | — | — |
| B | — | — | — | — | — | — |
| C | — | — | — | — | — | — |
| Comparative | | | | | | |
| Example 1 | | | | | | |
| A | — | — | — | 28 | 10 | — |
| B | — | — | — | 32 | 12 | — |
| C | — | — | — | 25 | 8 | — |
| Comparative | | | | | | |
| Example 1 | | | | | | |
| A | — | — | — | 34 | 47 | — |
| B | — | — | — | 25 | 42 | — |
| C | — | — | — | 22 | 35 | — |
| Example 1 | | | | | | |
| A | 14⁵⁾ | 5 | 1 | 13 | 20 | 0.61 |
| B | 10 | 3 | 2 | 10 | 17 | 0.56 |
| C | 9 | 2 | 3 | 8 | 12 | 0.70 |
| Example 2 | | | | | | |
| A | 12 | 8 | 3 | 11 | 20 | 0.74 |
| B | 10 | 5 | 3 | 10 | 22 | 0.56 |
| C | 7 | 3 | 2 | 6 | 14 | 0.60 |
| Example 3 | | | | | | |
| A | 2 | 2 | 1 | 13 | 5 | 0.28 |
| B | 3 | 2 | 1 | 15 | 4 | 0.32 |
| C | 2 | 1 | 1 | 14 | 3 | 0.23 |

¹⁾A: Panax ginseng B: Panax quinquefolia and C: Panax notoginseng
²⁾PPD: Protopanazadiol
³⁾DHPPD: 20-Dehydroprotopanaxadiol
⁴⁾Ratio: (Rk₂ + Rh₃ + PPD + DHPPD)/(Rg₃ + Rg₅ + Rk₁)
⁵⁾Concentration denotes a ratio of a area of each saponin peak to that of total saponin

As a result, nonpolar saponin components such as ginsenoside Rg₃, Rg₅, Rk₁, Rk₂, Rh₃, PPD and DHPPD in the sample of Comparative Example 1, were not detected, which shows that non-processed ginseng itself do not contains those saponins. However, Table 1 shows that the content of ginsenoside Rg₃ in the extract prepared by Comparative Example 2 are relatively higher than other components. In the extract prepared by Comparative Example 3, the amount of ginsenoside Rg₃, Rg₅ and Rk₁ are relatively higher than that of other components and ginsenoside Rk₂, Rh₃, PPD and DHPPD were not detected or merely detected. However, Table 1 shows that the extract prepared by Example 1, 2 and 3, contained high amount of ginsenoside Rk₂, Rh₃, PPD and DHPPD.

### Experimental Example 2:

Anticancer Activity Experiment

In order to confirm the anticancer effect of the extract of processed Panax genus plant in the present invention, the experiment was performed by the procedure described in the literature (Carmichael, J. et al., Cancer Res., 47, pp936-940, 1987).

Method

Liver cancer cell line (HepG2; Korean Cell Line Bank, Cat. No. 58065) was incubated at RPMI 1640 medium containing 10% FBS, 1% antibiotics-antimycotics (GIBCO, Cat. No. 15240-062) and 2.2 g/l of bicarbonate under 5% CO₂ gas condition. The cell was treated with 0.25% trypsin, separated from flask and adjusted the number of the cells to 3×10⁴ cells/well. 180 μl of each aliquot was transferred to 96 well plates, incubated in CO₂ incubator with 5% CO₂ gas for at 37° C., 24 hours and the cells were adhered to flask. Test sample solution containing the extract of Panax genus plant in Comparative Example 1, 2, 3 and Example 1, 2, 3, was then sterilized under high pressure vapor and prepared at various concentration. The samples were then added to 20 μl of each well and incubated at CO₂ incubator under 5% CO₂ gas for 48 hours. After 48 hours, 50 μl of MTT reagent solution at the concentration of 20 mg/ml (Sigma, Cat. No. M-5655) was added to each well, incubated in CO₂ incubator for 4 hours and the medium was removed. 100 μl of DMSO was added to the precipitate and the optical density was measured at 580 nm by ELISA reader (Bio Tek Instrument Inc., USA) to determine the inhibitory effect against cell growth.

GI₅₀ denotes the inhibitory concentration of sample to inhibit the cancer growth by 50% as described in Table 2.

Result

We confirmed that the anti-cancer effect of extracts in the Comparative Example 3 is more potent than those of the extract in the Comparative Example 1 and 2, however, the Example 1, 2 and 3 show more potent anticancer activities than that of Comparative Example 3. In summary, it is confirmed that the extract in the present invention shows most potent anticancer activity as can be seen in table 2.

TABLE 2
inhibitory effect of cancer cell growth (GI₅₀, material ginseng μg/ml)

| | Panax ginseng | Panax quinquefolia | Panax notoginseng |
|---|---|---|---|
| Comparative Example 1 | >5000 | >5000 | >5000 |
| Comparative Example 2 | 1500 | 2040 | 2580 |
| Comparative Example 3 | 720 | 1200 | 1320 |
| Example 1 | 240 | 300 | 390 |
| Example 2 | 310 | 370 | 400 |
| Example 3 | 480 | 580 | 600 |

### Experimental Example 3

Anti-Allergic Effect by using RBL-2H3 Cell Line

In order to confirm the anti-allergic effect of the extracts in the present invention, the experiment was performed by the procedure described in the literature (Inagaki. et al., Int, Arch. Allergy Appl. Immunol., 87, pp254-259, 1988).

Method

RBL-2H3 cell line (rat basophil cell line, Korean Cell Line Bank, Cat. No.22256) were incubated with DMEM (Dulbecco's modified Eagle's) medium (Sigma No. D-5648, USA) containing 10% FBS (Fetal Bovine Serum), L-glutamine for 2 hours at 37° C. by using humidified 5% CO₂ incubator and attached cells were floated by trypsin-EDTA solution, separated and recovered to use in further experiment. Each RBL-2H3 cells were transferred to 24 well plate to be adjusted to 5×10⁵ cells/well, treated with 0.5 μg of mouse monoclonal IgE and incubated for 12 hours to be sensitization. The cell was washed with 0.5 ml of siraganian buffer solution (pH 7.2) composed of 119 mM NaCl, 5 mM KCl, 0.4 mM MgCl₂, 25 mM PIPES and 40 mM NaOH. 0.16 ml of siraganian buffer solution containing 5.5 mM glucose, 1 mM CaCl₂ and 0.1% BSA, was added thereto again and incubated for 10 minutes at 37° C. Thereafter, 40 μl of test sample solution containing the extract in the Comparative Example 1, 2, 3 and Example 1, 2, 3 were added thereto. After 20 minutes, the cells were activated by adding 0.02 ml of antigen, 1 μg/ml of DNP-BSA, for 10 minutes at 37° C., centrifuged at the speed of 2000 rpm for 10 minutes and 0.025 ml of the supernatant was transferred to 96 well plate. 0.025 ml of 1 mM p-NAG substrate solution prepared by dissolving p-nitrophenyl-N-acetyl-β-D-glucosaminide with 0.1M citrate buffer solution adjusted to pH 4.5, was added thereto, incubated for 60 minutes at 37° C. and 0.2 ml of 0.1M Na₂CO₃/NaHCO₃ was added thereto to quench the reaction to measure their optical density at 405 nm by ELISA analyzer.

Result

The concentration of each sample to inhibit allergy by 50% (IC₅₀) is shown in Table 3. It is confirmed that the extract in Example 1, 2 and 3 exhibit more potent anti-allergic effect than those of Comparative Example 1, 2 and 3 by allergy inhibition test using RBL-2H3 cell line (Table 3). As can be seen from Table 3, the anti-allergic effect of present invention is proved to be most potent. Also, we confirmed that saponin fraction and the saponins prepared in Example 7 exhibit equivalent or more potent anti-allergic effect than that of the control drug such as DSCG (Disodium Cromoglycate, C-0399, Sigma, USA) by allergy inhibition test using RBL-2H3 cell line, in particular, it is clarify that ginsenoside Rh₁, Rh₂ and F1 show more potent inhibition effect than that of DSCG (Disodium Cromoglygate) as shown in Table 4.

TABLE 3
Anti-allergic effect in RBL cell (IC₅₀: material ginseng μg/ml)

| | Panax ginseng | Panax quinquefolia | Panax notoginseng |
|---|---|---|---|
| Comparative Example 1 | >50 | >50 | >50 |
| Comparative Example 2 | >50 | >50 | >50 |
| Comparative Example 3 | >50 | >50 | >50 |
| Example 1 | 12 | 17 | 22 |
| Example 2 | 15 | 19 | 21 |
| Example 3 | 24 | 28 | 29 |

TABLE 4
Anti-allergic effect in RBL cell

| Sample | IC₅₀ (μg/ml) |
|---|---|
| Saponin fraction (1) | >0.2 |
| Saponin fraction (2) | 0.1 |
| Ginseng fermented | 0.18 |
| Ginseng fermented | 0.15 |
| Ginsenoside Rb₁ | >0.2 |
| Ginsenoside Rb₂ | >0.2 |
| Ginsenoside Re | 0.06 |
| Ginsenoside Rg₁ | 0.08 |
| Ginsenoside Rf | 0.11 |
| 20(S)-ginsenoside Rg₃ | >0.2 |
| 20(R)-ginsenoside Rg₃ | >0.2 |
| Δ²⁰-ginsenoside Rg₃ | 0.09 |
| Ginsenoside F1 | 0.13 |
| 20(S)-ginsenoside Rh₂ | 0.03 |
| 20(R)-ginsenoside Rh₂ | >0.2 |
| Ginsenoside Rh₁ | 0.02 |
| 20(R)-protopanaxadiol | >0.2 |
| 20(S)-protopanaxadiol | >0.2 |
| DSCG (positive control) | 0.498 |

### Experimental Example 4

Passive Cutaneous Anaphylaxis Test

In order to confirm the anti-allergic effect of extracts in Comparative Example 1, 2 and Example 4, 5, 6 of the present invention, passive cutaneous anaphylaxis test was carried out.

Method

50 μl of diluted solution with physiological saline containing 10 μg of mouse IgE serum against dinitrophenol-human serum album (DNP-HSA, Sigma, USA), was injected to the back of ICR mouse (Dae Han Co., Ltd) anesthetized with ether to be passive sensitization. After 48 hours, 0.2 ml of physiological saline solution containing 0.2 mg of DNP-HSA and 1.6 mg of Evans blue was injected to the mouse's tail vein and the mouse was sacrificed by cervical dislocation to death and then the amount of Evans blue dye leaked to mouse's back was measured as follows. Certain circular area of mouse's back distant from the area where IGE injected about 1 cm² far, was cut and put into a test tube. 4 ml of mixture solution of 0.6M phosphoric acid and acetone with a ratio of 5:13 was added to the tube, stirred and filtered. The amount of dye was measured by colorimetric analysis at 620 nm. All the extracts of Comparative Example 1, 2 and in Example 4, 5, 6 were dissolved or suspended with physiological saline solution and administrated to the mouse orally 1 hour prior to the injection of DNP-HAS (antibody).

Result

It is confirmed that anti-allergic effect of the extracts in Example 4, 5 and 6 were very potent, while non-processed extract of Comparative Example 1 shows no anti-allergic effect and acid treated extract of the Comparative Example 2 shows low anti-allergic effect (Table 5). Also, we confirmed that saponin fraction and saponin compounds separated in the Example 7 exhibit equivalent or more potent anti-allergic effects than that of the control drug such as DSCG (Disodium Cromoglycate, C-0399, Sigma, USA) by anti-allergy test using RBL-2H3 cell line, in particular, it is clarify that ginsenoside Rh₁, Rh₂ and F1 show more potent inhibitory effects than that of DSCG (Table 6).

TABLE 5
Passive cutaneous anaphylaxis effect of Panax ginseng

| Sample | Dosage (mg/kg) | Inhibitory rate (%) |
|---|---|---|
| Comparative Example 1 | 50 | 0 |
| Comparative Example 2 | 50 | 20 |
| Example 4 | 50 | 52 |
| Example 5 | 50 | 62 |
| Example 6 | 50 | 54 |
| DSCG (positive control) | 100 | 37 |

TABLE 6
Passive cutaneous anaphylaxis effect

| | Dosage | Inhibitory rate (%) | |
|---|---|---|---|
| Sample | (mg/kg) | Oral administration | Abdominal administration |
| Saponin fraction (1) | 50 | 34 | — |
| Saponin fraction (2) | 50 | 47 | — |
| Ginsenoside Rb₁ | 50 | — | 17 |
| Ginsenoside Rb₂ | 25 | — | 12 |
| Ginsenoside Re | 25 | — | 15 |
| Ginsenoside Rg₁ | 25 | — | 37 |
| Ginsenoside Rf | 25 | — | 40 |
| Ginsenoside Rg₃ | | 38 | 47 |
| Gginsenoside Rg₅ | 25 | — | 45 |
| Ginsenoside F1 | 25 | — | 69 |
| Ginsenoside Rh₂ | 25 | 36 | 88 |
| Ginsenoside F4 | 25 | — | 63 |
| Ginsenoside Rh₁ | 25 | 79 | 87 |
| protopanaxadiol | 25 | — | 25 |
| DSCG (positive control) | 100 | 37 | |

As described above, it is confirmed that processed Panax genus plant prepared by the present invention showed more therapeutic and protective effect for cancer or allergy than that of non-processed plant and thus, it is useful for anti-cancer or anti-allergic drug or health care food.

### Experimental Example 5

Toxicity test

Methods (1)

The acute toxicity tests on ICR mice (mean body weight 25±5 g) and Sprague-Dawley rats (235±110 g, Hyochang Science) were performed using the extract of the Example 1. Four group consisting of 10 mice or rats was administrated orally intraperitoneally with 500 mg/kg, 725 mg/kg, 1000 mg/kg and 5000 mg/kg of test sample or solvents (0.2 ml, i.p.), respectively and observed for 2 weeks.

Methods (2)

The acute toxicity tests on ICR mice and Sprague-Dawley rats were performed using the extract of the Example 1. Four group consisting of 10 mice or rats was administrated intraperitoneally with 25 mg/kg, 250 mg/kg, 500 mg/kg and 725 mg/kg of test sample or solvents (0.2 ml, i.p.), respectively and observed for 24 hours.

Results

There were no treatment-related effects on mortality, clinical signs, body weight changes and gross findings in any group or either gender. These results suggested that the extract prepared in the present invention were potent and safe.

Hereinafter, the formulating methods and kinds of excipients will be described, but the present invention is not limited to them. The representative preparation examples were described as follows.

Preparation of Powder

| | |
|---|---|
| Dried powder of Example 1 | 50 mg |
| Lactose | 100 mg |
| Talc | 10 mg |

Powder preparation was prepared by mixing above components and filling sealed package.

Preparation of Tablet

| | |
|---|---|
| Ginsenoside Rh1 | 50 mg |
| Corn Starch | 100 mg |
| Lactose | 100 mg |
| Magnesium Stearate | 2 mg |

Tablet preparation was prepared by mixing above components and entabletting.

Preparation of Capsule

| | |
|---|---|
| Dried powder of Example 1 | 50 mg |
| Corn starch | 100 mg |
| Lactose | 100 mg |
| Magnesium Stearate | 2 mg |

Tablet preparation was prepared by mixing above components and filling gelatin capsule by conventional gelatin preparation method.

Preparation of Injection

| | |
|---|---|
| Ginsenoside Rh1 | 50 mg |
| Distilled water for injection | optimum amount |
| PH controller | optimum amount |

Injection preparation was prepared by dissolving active component, controlling pH to about 7.5 and then filling all the components in 2 ml ample and sterilizing by conventional injection preparation method.

Preparation of Liquid

| Dried powder of Example 1 | 0.1˜80 g |
|---|---|
| Sugar | 5˜10 g |
| Citric acid | 0.05˜0.3% |
| Caramel | 0.005˜0.02% |
| Vitamin C | 0.1˜1% |
| Distilled water | 79˜94% |
| CO₂ gas | 0.5˜0.82% |

Liquid preparation was prepared by dissolving active component, filling all the components and sterilizing by conventional liquid preparation method.

Preparation of Health Care Food

| Extract of Example 1 | 1000 | mg |
|---|---|---|
| Vitamin mixture | optimum amount | |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Amide nicotinic acid | 1.7 | mg |
| Folic acid | 50 | μg |
| Calcium pantothenic acid | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above mentioned vitamin and mineral mixture may be varied in many ways. Such variations are not to be regarded as a departure from the spirit and scope of the present invention.

Preparation of health beverage

| | | |
|---|---|---|
| Extract of Example 1 | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirred at 85° C. for 1 hour, filtered and then filling all the components in 1000 ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A processed Panax genus plant or the extract thereof comprising a ratio of ginsenoside (Rk₂+Rh₃+protopanaxadiol+20-dehydroprotopanaxadiol) to (Rg₃+Rg₅+Rk₁) of above 0.1.

2. The processed Panax genus plant or the extract of claim 1, wherein said ratio is above 0.2.

3. The processed Panax genus plant or the extract of claim 2, wherein said ratio is above 0.5.

4. A processed Panax genus plant or the extract thereof obtained by the steps essentially comprising acid or heat treating or their combination thereof the plant material belonged to Panax genus, and subsequent fermentation treating with lactic-acid bacteria or intestinal-bacteria.

5. The processed Panax genus plant or the extract thereof according to claim 4 wherein said Panax genus plant comprise at least one selected from the group consisting of Panax ginseng, Panax quinquefolia, Panax notoginseng, Panax japonica, Panax trifolia, Panax pseudoginseng, Panax vietnamensis, Panax elegatior, Panax wangianus, Panax bipinratifidus and Panax angustifolium.

6. The processed Panax genus plant or the extract thereof according to claim 4 wherein said plant material comprise the root, stem, petal, leaf, fruit and their tissue thereof.

7. The processed Panax genus plant or the extract thereof according to claim 4 wherein said plant material comprise fresh ginseng, processed ginseng and ginseng by-product thereof.

8. A process for preparing the Panax genus plant or the extract thereof as set forth in claim 1 by the steps essentially comprising: acid or heat treating or their combination thereof the plant material belonged to Panax genus; and subsequent fermentation treating with lactic-acid bacteria or intestinal-bacteria.

9. A process for preparing of pharmacologically active fractions or saponin compounds, comprising the steps of: subjecting the extract prepared by the process according to claim 8 to extraction or isolation process by lower alcohols to obtain partially purified fraction; and additionally, subjecting the fraction to Silica gel column chromatography to isolate saponin compounds therefrom.

10. The process according to claim 8 wherein said process comprises the steps of: subjecting dried plant material to acid treatment consisting of adding about 1 to 50 times v/v % of acidic component, incubating, neutralizing with base to obtain chemically modified Panax genus extract; and subsequently to fermentation step consisting of adding lactic acid bacteria or intestinal-bacteria to the extract, incubating to obtain processed extract.

11. The process according to claim 10 wherein said acid treatment is conducted by 0.1 to 10% acidic components selected from the group of acetic acid, citric acid, lactic acid and acidic tasting food.

12. The process according to claim 8 wherein said process comprises the steps of:
subjecting dried plant material to heat treatment at high temperature to obtain chemically modified Panax genus extract; and subsequent fermentation step consisting of adding lactic acid bacteria or intestinal-bacteria to the extract and incubating to obtain processed extract.

13. The process according to claim 12 wherein said heat treatment is conducted at the temperature ranging from 110 to 180° C.

14. The process according to claim 8 wherein said fermentation step is conducted by any bacteria which can metabolize ginsenoside Rg₃, Rg₅ and Rk₁ to ginsenoside Rh₂, Rh₃, Rk₂, protopanaxadiol and 20-dehydroprotopanaxadiol.

15. The process according to claim 12 wherein said lactic acid bacteria is belonged to Bifidobacterium or Lactobacilus.

16. The process according to claim 15 wherein said lactic acid bacteria comprises at least one or the mixture thereof selected from the group consisting of Bifidobacterium infantis, Bifidobacterium bifidum, Lactobacillus lactis, Clostridium butyricum, Bifidobacterium K-103, Bifidobacterium K-506, Bifidobacterium K-513, Bifidobacterium K-525, Bifidobacterium KK-1 and Bifidobacterium KK-2.

17. The process according to claim 12 wherein said intestinal-bacteria is belonged to Bacteriodes, Fusobacterium or Eubacterium.

18. The process according to claim 17 wherein intestinal-bacteria comprises at least one or the mixture thereof selected from the group consisting of Bacteriodes JY-6, Bacteriodes stercoris, Fusobacterium K-60 and Eubacterium L-8.

19. A pharmaceutical composition comprising processed extract as set forth in claim 1 to 3 as an active ingredient in an amount effective to treat or prevent human or mammal cancer diseases, together with a pharmaceutically acceptable carrier.

20. The pharmaceutical composition according to claim 19, wherein said cancer comprises liver cancer, lung cancer, skin cancer, ovarian cancer, blood cancer and uterine cancer.

21. A pharmaceutical composition comprising processed extract as set forth in claim 1 to 3 as an active ingredient in an amount effective to treat or prevent human or mammal allergic diseases, together with a pharmaceutically acceptable carrier.

22. A pharmaceutical composition comprising saponin fractions as set forth in claim 9 as an active ingredient in an amount effective to treat or prevent human or mammal in allergic diseases, together with a pharmaceutically acceptable carrier.

23. A pharmaceutical compositions comprising saponin compounds selected from the group consisting of ginsenoside Rb₁, Rb₂, Re, Rg₁, Rf, F1, F4, Rh₁, Rg₅, 20-ginsenoside Rg₃, 20-ginsenoside Rh₂, protopanaxadiol and the mixture thereof as an active ingredient in an amount effective to treat or prevent human or mammal in allergic diseases, together with a pharmaceutically acceptable carrier.

24. The pharmaceutical composition according to any of claims 21 to 23, wherein said allergic diseases comprise rhinitis, dermatitis, asthma and autoimmune deficiency disease.

25. The pharmaceutical composition according to any of claims 19 to 24, wherein said pharmaceutical composition is provided in an acceptable carrier as powder, granule, tablet, capsule, aqueous medicine or injection.

26. A method for treating or preventing cancer disease in a mammal comprising administrating to said mammal an effective amount of the extract as set forth in claim 1 to 3 or the saponin compounds selected from the group consisting of ginsenoside Rb₁, Rb₂, Re, Rg₁, Rf, F1, F4, Rh₁, Rg5, 20-ginsenoside Rg₃, 20-ginsenoside Rh2, protopanaxadiol and the mixture thereof and pharmaceutically acceptable carrier thereof.

27. A method for treating or preventing allergic disease in a mammal comprising administrating to said mammal an effective amount of the extract as set forth in claim 1 to 3 or the saponin compounds selected from the group consisting of ginsenoside Rb₁, Rb₂, Re, Rg₁, Rf, F1, F4, Rh₁, Rg5, 20-ginsenoside Rg₃, 20-ginsenoside Rh₂, protopanaxadiol and the mixture thereof and pharmaceutically acceptable carrier thereof.

28. A health care food comprising the extract as set forth in claim 1 to 3 and a sitologically acceptable additive to prevent cancer or allergic diseases.

29. The health care food according to claim 28, wherein said health care food is provided as beverage type.
